(19) United States
(12) Patent Application Publication
(10) Pub. No.: US 2008/0160062 A1
(43) Pub. Date: Jul. 3, 2008

(54) Medical devices based on modified polyols

(75) Inventor: Robert E. Richard, Wrentham, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 11/891,409

(22) Filed: Aug. 10, 2007

Related U.S. Application Data
(60) Provisional application No. 60/877,978, filed on Dec. 29, 2006.

(51) Int. Cl. A61F 2/06; A61F 2/00; A61K 47/30; C08F 20/04; C08G 63/48

(52) U.S. Cl. 424/423; 514/772.3; 525/57; 526/318.3; 528/425; 623/1.46

(57) ABSTRACT

According to an aspect of the present invention, implantable or insertable medical devices are provided, which contain at least one region of polymeric material. The at least one region of polymeric material contains at least one polymer selected from the following (a) at least one polymer that contains a polyol chain having charged groups along its length, (b) at least one polymer that contains a polyol chain having biodegradable polymer side chains along its length and (c) a combination thereof.

### STATEMENT OF RELATED APPLICATION

This application claims the benefit of U.S. Provisional Patent Application Ser. No. 60/877,978, filed Dec. 29, 2006, entitled “Medical Devices Based on Modified Polyols”, which is incorporated by reference herein.

### BACKGROUND OF THE INVENTION

A wide variety polymer-based medical devices have been developed for implantation or insertion into the body.

For example, in recent years, coronary stents, some of which release antiproliferative drugs at a controlled rate and total dose for preventing restenosis of the blood vessel, have become the standard of care for maintaining blood vessel patency. These products are based on metallic balloon expandable stents with polymeric coatings. For example, CYPHER (Johnson & Johnson, New Brunswick, N.J., USA) stents are coated with a thin layer of a blend of poly(n-butyl methacrylate) and ethylene-vinyl acetate copolymer and contain sirolimus as an anti-restenotic agent. R. Virmani et al., Circulation 2004 Feb. 17, 109(6) 701-5. The polymeric coating in the TAXUS (Boston Scientific Corp., Natick, Mass., USA) drug-eluting stent consists of a thermoplastic elastomer poly(styrene-b-isobutylene-b-styrene) (SIBS) with microphase-separated morphology resulting in optimal properties for a drug-delivery stent coating. S. Ranade et al, Journal of Biomedical Materials Research, 2004, 71A, 625.

As another example, permanent or temporary occlusion of blood vessels with polymer-based microspheres is desirable for managing various diseases, disorders and conditions. For instance, polymer-based microspheres are currently employed to embolize/occlude blood vessels to treat medical conditions such as hemorrhage and uterine fibroids, and as enhancements to chemotherapeutic treatment of tumors. Polymer-based microspheres are commonly introduced to the location of the intended embolization through microcatheters. Polymeric materials that have been used commercially include polyvinyl alcohol (PVA), acetalized PVA (e.g., Contour SE™ embolic agent, Boston Scientific Corp.) and crosslinked acrylic hydrogels (e.g., Embospheres®, Biosphere Medical, Rockland, Mass., USA). Similar devices have been used in chemoembolization procedures to increase the residence time of the therapeutic after delivery. In one specific instance, a therapeutic agent (doxorubicin) is directly added to polyvinyl alcohol copolymer hydrogel microspheres such that it can be released locally after delivery (e.g., DC Bead™ drug delivery chemoembolization system, A. Lewis et al., J. Vasc. Interv. Radiol., 2006, 17: 335-342). Microspheres are also used as bulking agents for cosmetic and therapeutic purposes.

### SUMMARY OF THE INVENTION

According to an aspect of the present invention, implantable or insertable medical devices are provided, which contain at least one region of polymeric material. The at least one region of polymeric material contains at least one polymer selected from the following (a) at least one polymer that contains a polyol chain having charged groups along its length, (b) at least one polymer that contains a polyol chain having biodegradable polymer side chains along its length and (c) a combination thereof.

These and various additional aspects, embodiments and advantages of the present invention will become immediately apparent to those of ordinary skill in the art upon review of the Detailed Description and Claims to follow.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

According to an aspect of the present invention, implantable or insertable medical devices are provided, which contain at least one region of polymeric material. The at least one region of polymeric material contains at least one polymer selected from the following (a) at least one polymer that contains a polyol chain having charged groups along its length, (b) at least one polymer that contains a polyol chain having biodegradable polymer side chains along its length and (c) a combination thereof.

As used herein, a “region of polymeric material” (also referred to herein as a “polymeric region”) is a material region that contains polymers. For example, a region of polymeric material in accordance with the present invention may contain from 50 wt % or less to 75 wt % to 90 wt % to 95 wt % to 97.5 wt % to 99 wt % or more polymers, as well as other optional agents such as therapeutic agents, among others.

In certain embodiments, the regions of polymeric material may correspond to entire medical devices. In certain embodiments, the regions of polymeric material may correspond to portions of medical devices.

For example, the region of polymeric material may be in the form of a polymeric layer covering all or only a portion of an underlying substrate (e.g., a metallic, ceramic, polymeric, etc. substrate), for example, a stent, among many other possibilities. As used herein a “layer” of a given material is a region of that material whose thickness is small compared to both its length and width. As used herein a layer need not be planar, for example, taking on the contours of an underlying substrate. Layers can be discontinuous (e.g., patterned).

As another example, the regions of polymeric material of the invention may be in the form of particles, for example, for use in embolic and tissue bulking procedures, among others. Such particles may take on a range of shapes. In certain embodiments, they are spherical, for example, having the form of a perfect (to the eye) sphere, or they are substantially spherical, for instance, in the form of a prolate spheroid (a slightly elongated sphere), an oblate spheroid (a slightly flattened sphere), and so forth. The injectable particles of the invention can be of various sizes, with typical longest linear cross-sectional dimensions (e.g., for a sphere, the diameter) ranging, for example, from 150 to 250 to 500 to 750 to 1000 to 1500 to 2000 to 2500 to 5000 microns (μm).

As used herein, “polymers” are molecules that contain multiple copies of one or more types of constitutional units, commonly referred to as monomers. As used herein, the term “monomers” may refer to the free monomers and those that are incorporated into polymers, with the distinction being clear from the context in which the term is used. The number of monomers/constitutional units within a given polymer may vary widely, ranging, for example, from 5 to 10 to 25 to 50 to 100 to 1000 to 10,000 or more constitutional units. Polymers for use in the region of polymeric materials of the present invention can have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains, such as graft polymers), dendritic architectures (e.g., arborescent and hyperbranched polymers), and networked architectures (e.g., crosslinked polymers), among others.

Polymers containing a single type of monomer are called homopolymers, whereas polymers containing two or more types of monomers are referred to as copolymers. The two or more types of monomers within a given copolymer may be present in any of a variety of distributions including random, statistical, gradient and periodic (e.g., alternating) distributions, among others. One particular type of copolymer is a “block copolymer,” which is a copolymer that contains two or more polymer chains of different composition, which chains may be selected from homopolymer chains and copolymer chains (e.g., random, statistical, gradient or periodic copolymer chains).

As used herein, a polymer “chain” is a linear assembly of monomers and may correspond to an entire polymer or to a portion of a polymer.

As used herein, a “polyol chain” is a polymer chain that comprises hydroxyl (—OH) groups along its length. Other groups, for example, acetate groups, cationic groups, anionic groups, and/or biodegradable groups, among other possibilities, are generally found along the polyol chains of the invention. Polymers comprising one or more polyol chains are referred to herein as “polyols.”

Polyol chains include homopolymer chains of a single monomer and copolymer chains of two or more monomers. A few specific examples of polyol chains include polyvinyl alcohol (PVA), poly(hydroxyalkyl acrylates) including poly(2-hydroxymethyl acrylate), poly(2-hydroxyethyl acrylate) and poly(2-hydroxypropyl acrylate), poly(hydroxyalkyl methacrylates) including poly(2-hydroxymethyl methacrylate), poly(2-hydroxyethyl methacrylate) and poly(2-hydroxypropyl methacrylate), polysaccharides including cellulose and cellulose derivatives, for example, hydroxyalkyl celluloses such as hydroxymethyl cellulose, hydroxyethyl cellulose and hydroxypropyl cellulose, and 4-hydroxystyrene, among others.

As discussed in more detail below, the polymers of the present invention may be crosslinked in various ways.

In certain embodiments, the regions of polymeric material of the present invention may optionally contain supplemental polymers other than polyols.

Medical devices benefiting from the present invention vary widely and include medical devices that are implanted or inserted into a subject, either for procedural uses or as implants.

Examples of medical devices include, for example, stents (including coronary vascular stents, peripheral vascular stents, cerebral, urethral, ureteral, biliary, tracheal, gastrointestinal and esophageal stents), catheters (e.g., renal or vascular catheters such as balloon catheters and various central venous catheters), guide wires, balloons, filters (e.g., vena cava filters and mesh filters for distil protection devices), stent grafts, vascular grafts, abdominal aortic aneurysm (AAA) devices such as AAA stents and AAA grafts, vascular access ports, dialysis ports, embolization devices including cerebral aneurysm filler coils (including Guglilmi detachable coils and metal coils), embolic agents, bulking agents, septal defect closure devices, myocardial plugs, patches, pacemakers, lead coatings including coatings for pacemaker leads, defibrillation leads and coils, ventricular assist devices including left ventricular assist hearts and pumps, total artificial hearts, shunts, valves including heart valves and vascular valves, anastomosis clips and rings, suture anchors, tissue staples and ligating clips at surgical sites, cannulae, metal wire ligatures, urethral slings, hernia “meshes”, artificial ligaments, orthopedic prosthesis, dental implants, cochlear implants, tissue bulking devices and agents, and tissue engineering scaffolds for cartilage, bone, skin and other in vivo tissue regeneration, biopsy devices, and any other device that is implanted or inserted into the body for medical purposes.

Medical devices benefiting from the present invention thus include a variety of implantable and insertable medical devices including devices for implantation or insertion into and/or through a wide range of tissues, organs and body lumens.

As noted above, the regions of polymeric material used in conjunction with the present invention contain at least one polymer selected from the following (a) at least one polymer that contains a polyol chain having charged groups along its length, (b) at least one polymer that contains a polyol chain having biodegradable polymer side chains along its length and (c) a combination thereof.

Medical devices useful for the treatment of a number of diseases, disorders and conditions can be constructed with such polymeric materials.

For instance, positively charged surfaces have been shown to lead to enhanced cellular attachment, relative to neutral or negatively charged surfaces.

Various studies have shown, for example, that positively charged substrates are conducive to endothelial cell adhesion and morphological maturation (flattening). See, e.g., G. L. Bowlin et al., “Electrostatic endothelial cell transplantation within small-diameter (<6 mm) vascular prostheses: a prototype apparatus and procedure,” Cell Transplant., November-December 1997, 6(6): 631-7 and the references cited therein.

P. B. van Wachem et al., “Adhesion of cultured human endothelial cells onto methacrylate polymers with varying surface wettability and charge,” Biomaterials, September 1987, 8(5): 323-8 studied the adhesion of human endothelial cells (HEC) onto a series of well-characterized methacrylate polymer surfaces with varying wettabilities and surface charges either in serum-containing (CMS) or in serum-free (CM) culture medium. Complete cell spreading in CM was only observed on the positively-charged copolymers.

H. Wang et al., “Modulating cell adhesion and spreading by control of FnIII7-10 orientation on charged self-assembled monolayers (SAMs) of alkanethiolates,” J Biomed Mater Res A, 15 Jun. 2006; 77(4): 672-8, demonstrated that surface charge can be used to modulate cell adhesion/spreading through the control of the orientation of adsorbed FnIII(7-10), a cell-adhesive protein containing arginine-glycine-aspartic acid (RGD) residues. Carboxylic acid (COOH) and amine (NH2)-terminated self-assembled monolayers of alkanethiolates were used as model negatively and positively charged surfaces, respectively. Adhesion/spreading of bovine aortic endothelial cells on the positively charged NH2—SAM was reported to be significantly better than that on the negatively charged COOH-SAM coated with an equivalent amount of FnIII(7-10), indicating that surface charge can be used to specifically orient cell adhesive proteins such as FnIII(7-10). Similarly, L. Liu et al., J Biomed Mater Res A, 2005 Jul. 1; 74(1): 23-31 investigated the effects of adsorption of osteopontin (OPN) (an extracellular matrix protein that contains the RGD moiety and has been shown to impact wound healing, inflammation, and the foreign body reaction) onto surfaces having self-assembled monolayers of alkanethiols terminated with various functional groups. They found that both endothelial cell count and average cell spreading area on a positively charged NH2 surface were much higher than those on a negatively charged COOH surface and they propose that the orientation and conformation of OPN on the positively charged NH2 surface is more favorable for cell adhesion and spreading than on the negatively charged COOH surface.

In “Biological responses to cationically charged phosphorylcholine-based materials in vitro,” Biomaterials, September 2004, 25(21): 5125-35, S. F. Rose et al. describe the affect of cationic charge on a range of biological responses to phosphorylcholine (PC)-based polymers. Specifically, in vitro assays were used to assess the adsorption of protein onto various surfaces, as well as the adhesion of mouse fibroblasts and rabbit corneal epithelial cells and the adhesion of human mononuclear cells and granulocytes. Their results corroborate previous work showing that PC without charge significantly reduces protein adsorption, cell adhesion and inflammatory cell activation. The addition of cationic charge to PC polymers however, resulted in an increase in all of theses responses.

K. W. Chun et al., “Biodegradable PLGA microcarriers for injectable delivery of chondrocytes: effect of surface modification on cell attachment and function,” Biotechnol Prog, November-December 2004, 20(6): 1797-1801, cultured bovine chondrocytes on three types of poly(D,L-lactic-co-glycolic acid) (PLGA) microspheres (i.e., those having hydrophobic, negatively charged, and positively charged surfaces) under serum conditions to comparatively evaluate cell attachment, cell proliferation, and cell function with respect to surface properties. Of these, positively charged PLGA microspheres showed the highest cell attachment, growth and function, compared to hydrophobic and negatively charged microspheres. Similarly, D Chen et al., Journal of Biomedical Engineering, [A study on cytocompatibility of poly (lactic acid) membrane modified by polymer microspheres with different surface charges], October 2005; 22(5): 966-70, report that the attachment, proliferation and activity of chondrocytes on poly(lactic acid) microspheres with a positively charged surface were better than those having other surface charges. Chondrocytes produce and maintain the extracellular matrix of cartilage, and autologous chondrocytes have been used as bulking agents for the treatment of intrinsic sphincter deficiency and vesicoureteral reflux. See, e.g., A. E. Bent et al., “Treatment of intrinsic sphincter deficiency using autologous ear chondrocytes as a bulking agent,” Neurourol. Urodyn., 2001; 20(2): 157-65 and D. A. Diamond et al., “Endoscopic correction of vesicoureteral reflux in children using autologous chondrocytes: preliminary results,” J. Urol., September 1999, 162(3 Pt 2): 1185-8.

Thus, positively charged polymers in accordance with the present invention may be employed in polymeric portions of devices upon which the attachment and growth of cells is desired. For example, positively charged polymers in accordance with the present invention may be employed in polymeric portions of devices for which endothelial cell attachment and growth is desired (e.g., stents, vascular grafts, valves, and embolic particles and coils, among others). As another specific example, positively charged polymers in accordance with the present invention may be employed in polymeric portions of devices for which chondrocyte attachment and growth is desired (e.g., bulking particles and orthopedic defect repair devices, among others). The devices may further optionally contain one or more therapeutic agents, for example, an RGD-containing agent, among many others.

As noted above, positively charged polymers have been observed to increase inflammatory cell activation. Moreover, various polyesters, including PLA and PLGA, are known to cause inflammation under certain circumstances. Such an effect may lead, for example, to enhanced bulking (e.g., due to scar tissue formation as a result of the inflammatory process) and to accelerated embolization (e.g., due to accelerating thrombosis/clotting), among other effects. Thus, polyols having positively-charged and/or biodegradable groups may be employed in polymeric portions of devices for which it is desirable to elicit an inflammatory response at the site of implantation (e.g., embolic compositions and bulking compositions, among others.

As another example, and as noted above, polyols having charged and/or biodegradable character may be used to locally deliver one or more therapeutic agents for various purposes, including the treatment of a number of diseases, disorders and conditions. “Therapeutic agents,” “drugs,” “pharmaceutically active agents,” “pharmaceutically active materials,” and other related terms may be used interchangeably herein. The presence of ionic and/or degradable groups allows multiple therapeutic agents to be released by various release mechanisms. These would include, for example, release based on cationic and/or anionic exchange mechanisms in the case of polyols with ionic character and release mediated by degradation in the case of polyols having biodegradable character.

Exemplary therapeutic agents for use in conjunction with the present invention include the following: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine) (u) bARKct inhibitors, (v) phospholamban inhibitors, (w) Serca 2 gene/protein, (x) immune response modifiers including aminoquizolines, for instance, imidazoquinolines such as resiquimod and imiquimod, and (y) human apolioproteins (e.g., AI, AII, AIII, AIV, AV, etc.).

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin (sirolimus) and its analogs (e.g., everolimus, tacrolimus, zotarolimus, etc.), cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference to the extent that is does not conflict with the present application.

Further therapeutic agents for use in the devices of the invention include therapeutic metals. For example positively charged radioactive metals such as yttrium, holmium, or phosphorous, among others, may be complexed/coordinated with anionic polyols within polymeric regions in accordance with the invention. For instance, a stent or other medical implant may be formed from or coated with such a polymeric region, thereby allowing for the release of such agents, for instance, in conjunction with brachytherapy.

As another example, metal ions that are capable of biocatalytic, in situ generation of nitric oxide, for instance, biomimetic catalytic agents such as Cu(II) as described in Pat. Pub. US 2002/0115559 to Batchelor et al., may be complexed/coordinated with anionic polyols within polymeric regions in accordance with the invention. For instance, a stent or other medical implant may be formed from or coated with such a polymeric region, thereby allowing for the release of nitric oxide. Nitric oxide has a number of known benefits including vaso-relaxation and accelerated healing, among others.

For embolic compositions in accordance with the present invention, examples of therapeutic agents to be used include toxins (e.g., a ricin toxin, a radionuclide, or any other agent able to kill undesirable cells such as those making up cancers and other tumors such as uterine fibroids) and agents that arrest growth of undesirable cells.

Some specific examples of therapeutic agents for embolic compositions (e.g., by providing them adsorbed to or within the embolic particles and/or within an fluid that suspends such particles) may be selected from suitable members of the following: antineoplastic/antiproliferative/anti-mitotic agents including antimetabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other aklyating agents (e.g., dacarbazine, etc.), antibiotics and analogs (e.g., daunorubicin, doxorubicin, idarubicin, mitomycin, bleomycins, plicamycin, etc.), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone), caspase activators, proteasome inhibitors, angiogenesis inhibitors (e.g., statins such as endostatin, cerivastatin and angiostatin, squalamine, etc.), rapamycin (sirolimus) and its analogs (e.g., everolimus, tacrolimus, zotarolimus, etc.), etoposides, as well as many others (e.g., hydroxyurea, flavopiridol, procarbizine, mitoxantrone, campothecin, etc.), and combinations of the foregoing, among other known antineoplastic/antiproliferative/anti-mitotic agents.

Various agents suitable for treatment of uterine fibroids (e.g., by providing them adsorbed to or within drug-releasing embolic particles and/or within an fluid that suspends such particles), many of which are suitable for the treatment of tumors other than fibroids, are listed in Ser. No. 11/124,828, which is hereby incorporated by reference to the extent that is does not conflict with the present application. These include chemical ablation agents (materials whose inclusion in the formulations of the present invention in effective amounts results in necrosis or shrinkage of nearby tissue upon injection) including osmotic-stress-generating agents (e.g., salts, etc.), basic agents (e.g., sodium hydroxide, potassium hydroxide, etc.), acidic agents (e.g., acetic acid, formic acid, etc.), enzymes (e.g., collagenase, hyaluronidase, pronase, papain, etc.), free-radical generating agents (e.g., hydrogen peroxide, potassium peroxide, etc.), other oxidizing agents (e.g., sodium hypochlorite, etc.), tissue fixing agents (e.g., formaldehyde, acetaldehyde, glutaraldehyde, etc.), coagulants (e.g., gengpin, etc.), non-steroidal anti-inflammatory drugs, contraceptives (e.g., desogestrel, ethinyl estradiol, ethynodiol, ethynodiol diacetate, gestodene, lynestrenol, levonorgestrel, mestranol, medroxyprogesterone, norethindrone, norethynodrel, norgestimate, norgestrel, etc.), GnRH agonists (e.g., buserelin, cetorelix, decapeptyl, deslorelin, dioxalan derivatives, eulexin, ganirelix, gonadorelin hydrochloride, goserelin, goserelin acetate, histrelin, histrelin acetate, leuprolide, leuprolide acetate, leuprorelin, lutrelin, nafarelin, meterelin, triptorelin, etc.), antiprogestogens (e.g., mifepristone, etc.), selective progesterone receptor modulators (SPRMs) (e.g., asoprisnil, etc.), and combinations of the foregoing, among other agents.

For tissue bulking applications (e.g., urethral bulking, cosmetic bulking, etc.), specific beneficial therapeutic agents include those that promote collagen production, including proinflammatory agents and sclerosing agents such as those listed in Ser. No. 11/125,297, which is hereby incorporated by reference to the extent that is does not conflict with the present application. For example, proinflammatory agents and/or sclerosing agents may be provided, for instance, within drug-releasing bulking particles and/or within a fluid that suspends such particles.

Suitable proinflammatory agents can be selected, for example, from suitable endotoxins, cytokines, chemokines, prostaglandins, lipid mediators, and other mitogens. Specific examples of proinflammatory agents from which suitable proinflammatory agents can be selected include the following: growth factors such as platelet derived growth factor (PDGF), fibroblast growth factor (FGF), transforming growth factor (such as TGF-alpha and TGF-beta), epidermal growth factor (EGF), insulinlike growth factor (IGF), interleukins such as IL-1-(alpha or beta), IL-8, IL-4, IL6, IL-10 and IL-13, tumor necrosis factor (TNF) such as TNF-alpha, interferons such as INF-gamma, macrophage inflammatory protein-2 (MIP-2), leukotrienes such as leukotriene B4 (LTB4), granulocyte macrophage-colony stimulating factor (GM-CSF), cyclooxygenase-1, cyclooxygenase-2, macrophage chemotactic protein (MCP), inducible nitric oxide synthetase, macrophage inflammatory protein, tissue factor, phosphotyrosine phosphates, N-formyl peptides such as formyl-Met-Leu-Phe (fMLP), second mitochondria-derived activator of caspase (sMAC), activated complement fragments (C5a, C3a), phorbol ester (TPA), superoxide, hydrogen peroxide, zymosan, bacterial lipopolysaccharide, chitin, imiquimod, carrageenan, and combinations of the foregoing, among other agents.

Suitable sclerosing agents for the practice of the invention can be selected, for example, from the following (which list is not necessarily exclusive of the pro-inflammatory list set forth above): inorganic materials such as talc, aluminum hydroxide (e.g., in slurry form), sodium hydroxide, silver nitrate and sodium chloride, as well as organic compounds, including alcohols such as ethanol (e.g., 50% to absolute), acetic acid, trifluoroacetic acid, formaldehyde, dextrose, polyethylene glycol ethers (e.g., polidocanol, also known as laureth 9, polyethylene glycol (9) monododecyl ether, and hydroxypolyethoxydodecane), tetracycline, oxytetracycline, doxycycline, bleomycin, triamcinolone, minocycline, vincristine, iophendylate, tribenoside, sodium tetradecyl sulfate, sodium morrhuate, diatrizoate meglumine, prolamine diatrizoate, alkyl cyanoacrylates such as N-butyl-2-cyanoactyalte and methyl 2-cyanoacrylate, ethanolamine, ethanolamine oleate, bacterial preparations (e.g., corynebacterium and streptococcal preparations such as picibanil) and mixtures of the same, for instance, TES (mixture of 1% tetradecyl sulfate, 32% ethanol, and 0.3% normal saline) and alcoholic solutions of zein (e.g., Ethibloc, which contains zein, alcohol, oleum papaveris, propylene glycol, and a contrast medium), and ethanol/trifluoroacetic acid mixtures, among others.

A wide range of therapeutic agent dosages can be used in conjunction with the medical devices of the present invention, with the therapeutically effective amount being readily determined by those of ordinary skill in the art based on various factors such as the disease, disorder or condition being treated, the potency of the therapeutic agent, and the mode of administration, among other factors.

Where a therapeutic agent is disposed on or within a polyol-containing region of polymeric material in accordance with the invention, typical loadings range, for example, from 1 wt % or less to 2 wt % to 5 wt % to 10 wt % to 25 wt % or more of the region of polymeric material.

The monomer of PVA (vinyl alcohol), does not exist in a stable free form, due to keto-enol rearrangement with its tautomer (acetaldehyde). Typically, PVA is produced by the polymerization of vinyl acetate to form poly(vinyl acetate) (PVAc), followed by hydrolysis of PVAc to PVA. The hydrolysis reaction, however, does not typically go to completion, resulting in polymers with a certain degree of hydrolysis that depends on the extent of reaction. Thus, PVA is generally a copolymer of vinyl alcohol and vinyl acetate. Commercial PVA grades are available with high degrees of hydrolysis (above 98.5%). The degree of hydrolysis (or, conversely, the acetate group content) of the polymer has an effect on its chemical properties, crystallizability, and solubility, among other properties. For example, degrees of hydrolysis and polymerization are known to affect the solubility of PVA in water, with PVA grades having high degrees of hydrolysis being known to have reduced solubility in water relative to those having low degrees of hydrolysis. Moreover, PVA grades containing high degrees of hydrolysis are more difficult to crystallize relative to those having low degrees of hydrolysis. For further information on PVA (as well as PVA hydrogels), see, e.g., C. M. Hassan et al., “Structure and Applications of Poly(vinyl alcohol) Hydrogels Produced by Conventional Crosslinking or by Freezing/Thawing Methods,” Adv. Polym. Sci., 153, 37-65 (2000) and N. A. Peppas et al., “Hydrogels in Biology and Medicine: From Fundamentals to Bionanotechnology”, Adv. Mater., 18, 1345-1360 (2006).

Polyvinyl alcohol (PVA) can be chemically modified to introduce anionic, cationic and degradable groups along its polymer chain. See, e.g., A. Breitenbach et al., “Biodegradable Comb Polyesters Containing Polyelectrolyte Backbones Facilitate the Preparation of Nanoparticles with Defined Surface Structure and Bioadhesive Properties,” Polym. Adv. Technol., Vol. 13, 938-950 (2002). As described in Breitenbach et al., sodium hydride may reacted with dimethylsulfoxide (DMSO) to created a DMSO carbanion according to the following scheme:

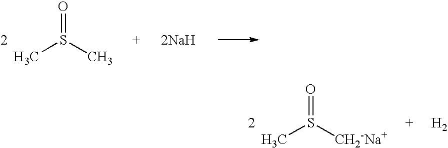

This DMSO carbanion is then added to PVA in dry DMSO to activate it:

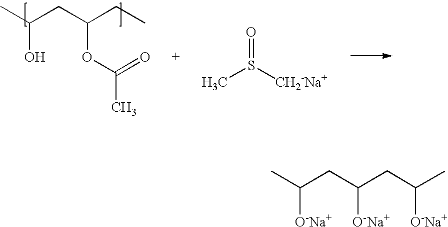

The activated PVA can then be reacted with 1,4-butanesultone

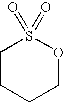

or with N-(2-chlorethyl)-N,N-diethylammonium-chloride

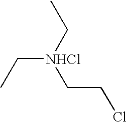

in dry DMSO, resulting in a sulfobutyl-modified PVA (SB-PVA) or in a diethylaminoethyl-modified PVA (DEAE-PVA), respectively, which are cationic and anionic, respectively, at neutral pH.

PVA, as well as the cationic SB-PVA and the anionic DEAE-PVA above, can each be converted to comb-type copolymers by grafting short polyester chains, including poly(lactic acid-co-glycolic acid) chains, onto the hydroxyl units of these polyols.

### Briefly, ring-opening bulk melt polymerization of the lactones,

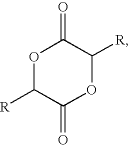

where R═H corresponds to the glycolic acid monomer and R═CH₃ corresponds to the lactic acid monomer, can be conducted in the presence of each of these polyols, using stannous octoate as catalyst.

PVA hydrogels can be formed by various physical and chemical techniques. A hydrogel can be described as a hydrophilic, crosslinked polymer (e.g., a polymer network) which swells when placed in water or biological fluids, but remains insoluble due to the presence of physical and/or chemical crosslinks. In some instances, the insolubility is not permanent. For example, physically crosslinked PVA hydrogels are known to be biodegradable.

PVA can be crosslinked, for example, through the use of difunctional crosslinking agents. Some of the common chemical crosslinking agents that have been used for PVA hydrogel preparation include glutaraldehyde, acetaldehyde, formaldehyde, and other monoaldehydes. In the presence of an acid such as sulfuric acid or acetic acid, these crosslinking agents form acetal bridges between the pendant hydroxyl groups found on the PVA chains. For example, acetal formation may proceed to link two alcohol moieties together according to the following scheme:

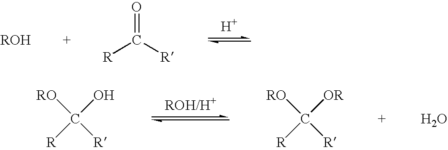

where R and R′ are organic groups. For species with multiple hydroxyl groups, including polyols, two hydroxyl groups within the same molecule may react according to the following scheme:

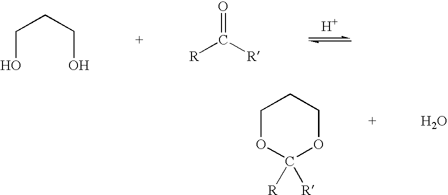

As noted in Pub. No. US 2003/0185895 to Lanphere et al., in certain instances, the reaction of PVA with an aldehyde (formaldehyde) in the presence of an acid is primarily a 1,3 acetalization:

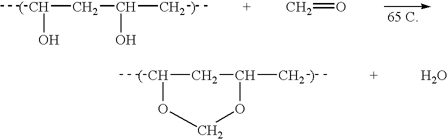

Such intra-chain acetalization reaction can be carried out with relatively low probability of inter-chain crosslinking. Since the reaction proceeds in a random fashion, there will be left over —OH groups that do not react with adjacent groups.

Other methods of chemical crosslinking polyols include the use of electron-beam and gamma-ray irradiation. These methods, as well as the physical crosslinking techniques described below, may in some instances be advantageous over techniques that employ chemical cross-linking agents, because they do not leave behind unreacted chemical species.

Other mechanisms of hydrogel preparation involve physical crosslinking due to crystallite formation. Such physically crosslinked materials have been reported to exhibit higher mechanical strength than chemically crosslinked PVA hydrogels, reportedly due to the fact that a mechanical load can be distributed along the crystallites of the three-dimensional structure. Aqueous PVA solutions have an unusual characteristic in that they form crystallites upon repeated freezing and thawing cycles. The number and stability of these crystallites are increased as the number of freezing/thawing cycles is increased.

Various techniques are thus available for forming regions of polymeric materials in accordance with the present invention.

As a specific example, microspheres of polyol suitable for injection (e.g., for embolic, bulking or other purposes) can be prepared by dispersing an aqueous polyol solution in an immiscible solvent and then crosslinking it with a suitable material such as an aldehyde.

Particles suitable for injection may also be formed as described in Pub. No. US 2003/0185895 to Lanphere et al. Briefly, a solution containing a polyol and a gelling precursor such as sodium alginate may be delivered to a viscosity controller, which heats the solution to reduce its viscosity prior to delivery to a drop generator. The drop generator forms and directs drops into a gelling solution containing a gelling agent which interacts with the gelling precursor. For example, in the case where an alginate gelling precursor is employed, an agent containing a divalent metal cation such as calcium chloride may be used as a gelling agent, which stabilizes the drops by gel formation based on ionic crosslinking. A pore structure in the center of the particle has been observed to form in the gelling stage. The concentration of the gelling agent can control void formation in the embolic particle, thereby controlling the porosity gradient in the embolic particle. Adding non-gelling ions, for example, sodium ions, to the gelling solution can limit the porosity gradient, resulting in a more uniform intermediate porosity throughout the particle. The gel-stabilized drops may then be transferred to a reactor vessel, where the polymer in the gel-stabilized drops are reacted, thereby forming precursor particles. For example, the reactor vessel may include an agent that chemically reacts with the polyol to cause interchain or intrachain crosslinking. For example, the vessel may include an aldehyde and an acid, leading to acetalization of the polyol. The precursor particles are then transferred to a gel dissolution chamber, where the gel is dissolved. For example, ionically crosslinked alginate may be removed by ion exchange with a solution of sodium hexa-metaphosphate. Alginate may also be removed by radiation degradation. The particles may then be filtered to remove any residual debris and to sort the particles into desired size ranges. The filtered particles may then be sterilized (e.g., by e-beam irradiation) and packaged, typically in saline.

Although chemical crosslinking is described in Lanphere et al., other crosslinking techniques such as crosslinking by irradiation or by repeated freezing and thawing may be employed.

Using analogous techniques, polyol-containing polymeric regions may be provided in a variety of forms other than particulate forms. For example, a solution containing a polyol may be applied to a substrate, for example, a medical device substrate (e.g., a stent) or a temporary substrate (e.g., a mold) using an appropriate technique (e.g., spraying, spin coating, web coating, dipping, solvent casting, techniques involving coating via mechanical suspension including air suspension, ink jet techniques, electrostatic techniques, and combinations of these processes). In some embodiments, the polyol-containing solution may also contain a gelling precursor, in which case a solution containing a gelling agent, which interacts with the gelling precursor, may also be applied to the substrate, for example, at the same time as the polyol-containing solution or in alternating steps with the polyol-containing solution. Once a polyol-containing region is established on the substrate, it may be then be crosslinked, for example, by contacting the polyol-containing region with a suitable crosslinking solution such as acidic aldehyde solution (e.g., by immersion, spraying, etc.), by irradiating the polyol-containing region or by subjecting the polyol-containing region to repeated freeze thaw cycles. Where the polyol-containing region is gel stabilized, the gel may then be dissolved. For example, ionically crosslinked alginate may be removed by ion exchange as described above. Alginate may also be removed by radiation degradation.

The polyol may be provided with charged and/or biodegradable groups before or after processing it into its final form (e.g., into the form of particles, into the form of a coating layer, etc.).

For example, a crosslinked polyol-containing polymeric region (e.g., a particle, coating, etc.) may be modified to introduce cationic, anionic and/or biodegradable groups as described above. As a specific example, Contour SE microspheres are commercially available from Boston Scientific Corp., Natick, Mass., USA. These particles are formed from acetalized PVA, which contains unreacted vinyl alcohol units, and it is these unreacted units that may be modified as above. Of course, other materials may be chosen for modification. Examples would include other forms of PVA (e.g., physically crosslinked or chemically crosslinked by methods other than acetalization) and materials formed from other polyols besides PVA, for example, poly(hydroxyalkyl acrylates), poly(hydroxyalkyl methacrylates) and polysaccharides, among others. Cationic, anionic and/or biodegradable groups may be selectively formed at the surface of the crosslinked polyol-containing polymeric region, for example, by selectively by using materials that are non-porous or using materials in which the reactive hydroxyl groups are made to exist primarily on the surface.

As another example, a polyol such as PVA may be chemically modified to provide it with cationic, anionic and/or biodegradable groups prior to providing it in its final form (e.g., in the form of a particle, coating, etc.). Where the polyol is stabilized by acetalization, processing in this manner would reduce the theoretical level of acetalization possible, but this may be offset by proper polyol selection. For example, where PVA is used as the polyol, PVA grades with a high degree of hydrolysis may be selected. Processing in this manner would allow different types of polyols to be blended with one another. For example, any combination of two or more of the following may be blended together: (a) polyol chemically modified to contain anionic groups, (b) polyol chemically modified to contain cationic groups, and (c) polyol chemically modified to contain biodegradable polyester groups.

If desired, one or more optional agents such as therapeutic agents can be incorporated at various stages. Depending on the nature of the therapeutic agents, they may ultimately be released via various release mechanisms, for example, cationic and/or anionic exchange, release mediated by degradation of polyester side chains, or diffusion-based release governed by the primary sphere material (e.g., acetalized PVA). For example, a crosslinked, polyol-containing polymeric region (e.g., particle, coating, etc.) can be dried and then rehydrated in a solution that includes therapeutic agent. In the rehydration process, the therapeutic agent is drawn into the polymeric region. The device may then be packed in a solution of therapeutic agent if desired.

Alternatively, the therapeutic agent may be added during formation of the polyol-containing polymeric region. As a specific example, therapeutic agent may be mixed with polyol and alginate prior to gel formation, among numerous other possibilities.

Although various embodiments are specifically illustrated and described herein, it will be appreciated that modifications and variations of the present invention are covered by the above teachings and are within the purview of the appended claims without departing from the spirit and intended scope of the invention.

For instance, many of the descriptions herein focus on the use of PVA as a polyol for explanatory purposes. However, as noted above, other polyols are useful in the practice of the present invention. Moreover, many of the descriptions herein focus upon stent coatings and particles, thereby illustrating that the polyol-containing polymeric regions of the invention can vary widely in shape, size and nature. However, the present invention is not so limited.

1. An implantable or insertable medical device comprising a region of polymeric material that comprises a polymer selected from the following: a polymer comprising a polyol chain that comprises charged groups along its length, a polymer comprising a polyol chain that comprises biodegradable polymer side chains along its length, and a combination thereof.

2. The implantable or insertable medical device of claim 1, wherein said polyol chain is polyvinyl alcohol chain.

3. The implantable or insertable medical device of claim 1, wherein said polyol chain is selected from a poly(hydroxyalkyl acrylate) chain and a poly(hydroxyalkyl methacrylate) chain.

4. The implantable or insertable medical device of claim 1, wherein said polyol chain is a cellulose chain or a cellulose derivative chain.

5. The implantable or insertable medical device of claim 1, wherein said region of polymeric material is crosslinked.

6. The implantable or insertable medical device of claim 1, wherein said region of polymeric material is chemically crosslinked.

7. The implantable or insertable medical device of claim 1, wherein said region of polymeric material is acetalized.

8. The implantable or insertable medical device of claim 1, wherein said region of polymeric material is radiation crosslinked.

9. The implantable or insertable medical device of claim 1, wherein said region of polymeric material is physically crosslinked.

10. The implantable or insertable medical device of claim 1, wherein the region of polymeric material comprises a polymer that comprises a polyol chain that comprises charged groups along its length.

11. The implantable or insertable medical device of claim 10, wherein said charged groups are cationic.

12. The implantable or insertable medical device of claim 11, wherein the region of polymeric material further comprises an anionic therapeutic agent.

13. The implantable or insertable medical device of claim 10, wherein the polyol chain comprises sulfobutyl groups.

14. The implantable or insertable medical device of claim 10, wherein said charged groups are anionic.

15. The implantable or insertable medical device of claim 14, wherein the region of polymeric material further comprises a cationic therapeutic agent.

16. The implantable or insertable medical device of claim 10, wherein the polyol chain comprises diethylaminoethyl groups.

17. The implantable or insertable medical device of claim 1, wherein the region of polymeric material comprises a polymer that comprises a polyol chain that comprises biodegradable polymer side chains along its length.

18. The implantable or insertable medical device of claim 17, wherein said biodegradable polymer side chains are biodegradable polyester side chains.

19. The implantable or insertable medical device of claim 18, wherein said biodegradable polyester is selected from homopolymer and copolymer side chains that comprise lactic acid monomers, glycolic acid monomers, or a combination thereof.

20. The implantable or insertable medical device of claim 1, comprising two or more regions of polymeric material.

21. The implantable or insertable medical device of claim 1, further comprising a therapeutic agent.

22. The implantable or insertable medical device of claim 1, further comprising a plurality of differing therapeutic agents.

23. The implantable or insertable medical device of claim 1, wherein said region of polymeric material is in the form of a particle.

24. The implantable or insertable medical device of claim 23, wherein said medical device selected from an embolic particle and a bulking particle.

25. The implantable or insertable medical device of claim 1, wherein said region of polymeric material is in the form of a coating on an underlying medical device.

26. The implantable or insertable medical device of claim 25, wherein said medical device is a stent.
